(19) United States
(12) Patent Application Publication
(10) Pub. No.: US 2025/0249230 A1
(43) Pub. Date: Aug. 7, 2025

(54) LEFT VENTRICAL ASSIST DEVICE AND COMPONENTS AND METHODS

(71) Applicants: David Melander, St. Louis Park, MN (US); Joseph Wang, Henderson, NV (US)

(72) Inventors: David Melander, St. Louis Park, MN (US); Joseph Wang, Henderson, NV (US)

(21) Appl. No.: 19/011,198

(22) Filed: Jan. 6, 2025

Related U.S. Application Data
(60) Provisional application No. 63/618,287, filed on Jan. 6, 2024.

(51) Int. Cl. A61M 60/178; A61M 60/216; A61M 60/422

(52) U.S. Cl. CPC A61M 60/178; A61M 60/216; A61M 60/422

(57) ABSTRACT

Embodiments herein relate to a left ventricle assist device (LVAD), the LVAD is included having an inflow port; an outflow port; an impeller configured to pump blood from the inflow port to the outflow port so as to assist the left ventricle of a heart in pumping blood; and a motor driving the impeller, the motor is included having a stator element; a rotating disc element; wherein the rotating disc element includes a polymagnet. Other embodiments are also included herein.

### FIELD

Embodiments herein relate to left ventricle assist devices (LVADs), including to components used in LVADS and methods of making and using LVADs.

### BACKGROUND

A left ventricular assist device (LVAD) is a pump typically used to treat patients who have reached end-stage heart failure. LVADs are typically battery-operated mechanical pumps that are typically implanted into a patient and help the left ventricle pump blood to the rest of the body. LVADs can be used as a temporary bridge to a heart transplant when recipients use the LVAD until a heart becomes available. LVADs can also be used to restore function in a failing heart, eliminating the need for a transplant, at least temporarily. This is especially useful because some patients are not candidates for heart transplants. In this case, patients can receive long-term treatment using an LVAD, which can prolong and improve their lives.

Heart failure is a complex clinical syndrome resulting from any structural or functional impairment of ventricular filling or ejection of blood and affects more than 6 million Americans. Advanced heart failure is defined as the presence of refractory symptoms despite optimal medical, surgical, and device therapy. It is estimated that approximately half a million individuals have late state heart failure in the United States, with millions more in other countries. Approximately 10 to 15 percent of these patients are eligible for advanced therapies yearly, but only a small percent receive an LVAD or heart transplant, leaving a large percentage of eligible patients deprived of these life-saving therapies. Also, despite efforts to increase the donor pool, heart transplant remains limited by the scarcity of donor hearts. However, LVAD therapy does not share the same constraints.

Although LVADs are useful and can save lives, improve health, and prolong lives, there remains a need for improved LVAD designs.

### SUMMARY

LVADs are often charactered as continuous-flow or pulsatile. Continuous-flow LVADs are smaller and have proven to be more durable than pulsatile LVADs. They normally use either a centrifugal pump or an axial flow pump. Both types have a central rotor containing permanent magnets. Controlled electric currents running through coils contained in the pump housing apply forces to the magnets, which in turn cause the rotor or rotors to spin. In the centrifugal pumps, the rotors are shaped to accelerate the blood circumferentially and thereby cause it to move toward the outer rim of the pump, whereas in the axial flow pumps the rotors are more or less cylindrical with blades that are helical, causing the blood to be accelerated in the direction of the rotor's axis.

Benefits of LVADs made using polymagnets include reduction of mass by: (a) dramatic reduction of mass of the permanent rare earth; (b) reduction of size of windings, coils; and (c) the ability to make general reductions of all materials. In addition, there can be a reduction in power consumption, allowing greater battery life and reduction in size of external power management systems. Greater proximities of coils to specific field locations on the magnet allows stronger pull forces which allow changes in geometries in the design, allowing more impeller features on the magnet. Further, polymagnets allow a magnet construction with additional features that allow placement of attract and repel forces in close proximity to the coils. It is also possible to reduce blood sheer and resulting thrombus and ischemic events (primarily stroke). Finally, the impeller features can be incorporated into the magnet, thereby reducing part mass.

The present application utilizes magnets, also referred to as a polymagnet, which are magnetic structures that incorporate correlated patterns of magnets with alternating polarity, designed to achieve a desired behavior and deliver stronger local force than alternative magnets. By varying the magnetic fields and strengths, different mechanical behaviors can be controlled.

In an embodiment, a left ventricle assist device (LVAD) is disclosed, the LVAD having an inflow port, an outflow port, an impeller configured to pump blood from the inflow port to the outflow port so as to assist the left ventricle of a heart in pumping blood, and a motor driving the impeller, the motor is included having a stator element, a rotating disc element, wherein the rotating disc element includes a polymagnet.

In an embodiment, the rotating disc element includes a polymagnet configured for levitation.

In an embodiment, the rotating disc element includes a polymagnet configured for application of rotational force to the rotating disc.

In an embodiment, the rotating disc element includes a polymagnet configured for application of rotational force to the rotating disc.

In an embodiment, the rotating disc element includes a polymagnet configured for application of rotational force to the rotating disc.

In an embodiment, the rotating disc includes a polymagnet configured for application of rotational force and levitating force to the rotating disc.

In an embodiment, the rotating disc includes a polymagnet configured for application of rotational force and levitating force to the rotating disc, wherein the same a polymagnet is used for rotational force and levitating force.

In an embodiment, the rotating disc includes a polymagnet configured for application of rotational force and levitating force to the rotating disc, wherein the polymagnet for the rotational force are located radially distinct from the polymagnet for levitation relative to the center of the disc.

In an embodiment, the rotating disc includes a polymagnet configured for application of rotational force and levitating force to the rotating disc, wherein the polymagnet for the rotational force are located concentrically outside relative to the polymagnet for levitation.

In an embodiment, the rotating disc includes a polymagnet configured for application of rotational force, wherein the polymagnet include alternating positive and negative magnetic regions.

In an embodiment, the rotating disc includes a polymagnet configured for application of levitational force, wherein the polymagnet include continuous concentric regions of positive or negative magnetic polarity.

In an embodiment, the polymagnet include sintered NiCuNi material.

In an embodiment, the polymagnet include NdFeB material.

In an embodiment, the polymagnet include sintered SmCo material.

In an embodiment, the polymagnet include a PTFE material.

In an embodiment, the rotating disc includes an open interior for passage of blood, the open interior having an area at least 10 percent of the area within the circumference of the disc.

In an embodiment, the rotating disc includes an open interior for passage of blood, the open interior having an area at least 15 percent of the area within the circumference of the disc.

In an embodiment, the rotating disc includes an open interior for passage of blood, the open interior having an area at least 20 percent of the area within the circumference of the disc.

In an embodiment, the rotating disc includes an open interior for passage of blood, the open interior having an area at least 25 percent of the area within the circumference of the disc.

In an embodiment, the rotating disc includes an open interior for passage of blood, the open interior having an area at least 30 percent of the area within the circumference of the disc.

In an embodiment, the rotating disc includes an open interior for passage of blood, the open interior having an area at least 35 percent of the area within the circumference of the disc.

In an embodiment, the rotating disc includes an open interior for passage of blood, the open interior having an area at least 40 percent of the area within the circumference of the disc.

In an embodiment, the rotating disc includes an open interior for passage of blood, the open interior having an area at least 45 percent of the area within the circumference of the disc.

In an embodiment, the rotating disc includes an open interior for passage of blood, the open interior having an area at least 50 percent of the area within the circumference of the disc.

In an embodiment, the rotating disc includes an open interior for passage of blood, the open interior having an area at least 60 percent of the area within the circumference of the disc.

In an embodiment, the rotating disc includes an open interior for passage of blood, the open interior having an area at least 70 percent of the area within the circumference of the disc.

In an embodiment, wherein clearance above and below the rotating disc relative to adjacent components is less than 3 millimeters.

In an embodiment, wherein clearance above and below the rotating disc relative to adjacent components is less than 2 millimeters.

In an embodiment, wherein clearance above and below the rotating disc relative to adjacent components is less than 1 millimeter.

In an embodiment, wherein clearance above and below the rotating disc relative to adjacent components is less than 0.5 millimeters.

In an embodiment, the rotor has a thickness of less than 5 millimeters.

In an embodiment, the rotor has a thickness of less than 4 millimeters.

In an embodiment, the rotor has a thickness of less than 3 millimeters.

In an embodiment, the rotor has a thickness of less than 2 millimeters.

In an embodiment, the thickness of the polymagnet are less than 2 millimeters.

In an embodiment, the thickness of the polymagnet is less than 1 millimeter.

In an embodiment, the polymagnet are arranged in a radially symmetric pattern.

In an embodiment, the polymagnet cover at least 50 percent of the surface area of the rotor.

In an embodiment, the polymagnet cover at least 60 percent of the surface area of the rotor.

In an embodiment, the polymagnet cover at least 70 percent of the surface area of the rotor.

In an embodiment, the polymagnet cover at least 80 percent of the surface area of the rotor.

In an embodiment, the polymagnet cover at least 90 percent of the surface area of the rotor.

In an embodiment, the polymagnet cover at least 95 percent of the surface area of the rotor.

The present application is directed, in part, to LVADs that incorporate magnets having preferred design features, including discs having field emission structures comprising a plurality of electric or magnetic field sources having magnitudes, polarities, and positions corresponding to a desired spatial force function where a spatial force is created based upon the relative alignment of a field emission structure and a complementary field emission structure. Specific exemplary embodiments are described with magnetic field sources arranged in a ring structure. The ring structure may include one or more concentric rings of component magnets. Mechanical constraints may be employed to limit lateral motion.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a simplified cross-sectional view of conventional left ventricle assist device.

FIG. 2 is a simplified cross-sectional view of a conventional left ventricle assist device.

FIG. 3 is a cross-sectional schematic view of elements of left ventricle assist device in accordance with various embodiments herein.

FIG. 4 is a perspective view of magnetic disc in accordance with various embodiments herein.

FIG. 5 is a top plan view of magnetic disc in accordance with various embodiments herein.

FIG. 6 is a top plan view of magnetic disc in accordance with various embodiments herein.

FIG. 7 is a top plan view of magnetic disc in accordance with various embodiments herein.

FIG. 8 is a top plan view of magnetic disc in accordance with various embodiments herein.

FIG. 9 is a top plan view of magnetic disc in accordance with various embodiments herein.

FIG. 10 is a top plan view of magnetic disc in accordance with various embodiments herein.

FIG. 11 is a top plan view of magnetic disc in accordance with various embodiments herein.

FIG. 12 is a top plan view of magnetic disc during manufacturing in accordance with various embodiments herein.

FIG. 13 is a cross-sectional schematic view of elements of left ventricle assist device in accordance with various embodiments herein.

FIG. 14 cross-sectional schematic view of elements of left ventricle assist device in accordance with various embodiments herein.

FIG. 15 are illustrations showing coil forces applied during operation of an LVAD in accordance with various embodiments herein.

FIG. 16 is a perspective view of left ventricle assist device in accordance with various embodiments herein.

FIG. 17 is an exploded perspective view of left ventricle assist device in accordance with various embodiments herein.

FIG. 18 is a perspective view of the impeller for a left ventricle assist device in accordance with various embodiments herein.

FIG. 19 is a perspective view of portions of a magnetic disc 1915 in accordance with various embodiments herein, showing coils 1916. In practice the coils 1916 can be flush with the surrounding surface of the disc.

FIG. 20 is a perspective view of example coils from a magnetic disc in accordance with various embodiments therein, showing the coils 2016 formed of wire 2017 with a square or rectangular cross section.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

In an embodiment, a left ventricle assist device (LVAD), the LVAD is included having an inflow port, an outflow port, an impeller configured to pump blood from the inflow port to the outflow port so as to assist the left ventricle of a heart in pumping blood, and a motor driving the impeller, the motor is included having a stator element, a rotating disc element, wherein the rotating disc element includes a plurality of a polymagnet.

In an embodiment, the rotating disc element includes a polymagnet configured for levitation. In an embodiment, the rotating disc element includes a polymagnet configured for application of rotational force to the rotating disc. In an embodiment, the rotating disc element includes a polymagnet configured for application of rotational force to the rotating disc. In an embodiment, the rotating disc element includes a polymagnet configured for application of rotational force to the rotating disc. In an embodiment, the rotating disc includes a polymagnet configured for application of rotational force and levitating force to the rotating disc.

In an embodiment, the rotating disc includes a polymagnet configured for application of rotational force and levitating force to the rotating disc, wherein the same a polymagnet are used for rotational force and levitating force. In an embodiment, the rotating disc includes a polymagnet configured for application of rotational force and levitating force to the rotating disc, wherein the polymagnet for the rotational force are located radially distinct from the polymagnet for levitation relative to the center of the disc.

In an embodiment, the rotating disc includes a polymagnet configured for application of rotational force and levitating force to the rotating disc, wherein the polymagnet for the rotational force are located concentrically outside relative to the polymagnet for levitation.

In an embodiment, the rotating disc includes a polymagnet configured for application of rotational force, wherein the polymagnet include alternating positive and negative magnetic regions. In an embodiment, the rotating disc includes a polymagnet configured for application of levitational force, wherein the polymagnet include continuous concentric regions of positive or negative magnetic polarity.

In an embodiment, the polymagnet include sintered NiCuNi material. In an embodiment, the polymagnet include NdFEB material. In an embodiment, the polymagnet include sintered SmCo material. In an embodiment, the polymagnet include a PTFE material.

In an embodiment, the rotating disc includes an open interior for passage of blood, the open interior having an area at least 10 percent of the area within the circumference of the disc. In an embodiment, the rotating disc includes an open interior for passage of blood, the open interior having an area at least 15 percent of the area within the circumference of the disc. In an embodiment, the rotating disc includes an open interior for passage of blood, the open interior having an area at least 20 percent of the area within the circumference of the disc. In an embodiment, the rotating disc includes an open interior for passage of blood, the open interior having an area at least 25 percent of the area within the circumference of the disc. In an embodiment, the rotating disc includes an open interior for passage of blood, the open interior having an area at least 30 percent of the area within the circumference of the disc. In an embodiment, the rotating disc includes an open interior for passage of blood, the open interior having an area at least 35 percent of the area within the circumference of the disc. In an embodiment, the rotating disc includes an open interior for passage of blood, the open interior having an area at least 40 percent of the area within the circumference of the disc. In an embodiment, the rotating disc includes an open interior for passage of blood, the open interior having an area at least 45 percent of the area within the circumference of the disc. In an embodiment, the rotating disc includes an open interior for passage of blood, the open interior having an area at least 50 percent of the area within the circumference of the disc. In an embodiment, the rotating disc includes an open interior for passage of blood, the open interior having an area at least 60 percent of the area within the circumference of the disc. In an embodiment, the rotating disc includes an open interior for passage of blood, the open interior having an area at least 70 percent of the area within the circumference of the disc.

In an embodiment, wherein clearance above and below the rotating disc relative to adjacent components is less than 3 millimeters. In an embodiment, wherein clearance above and below the rotating disc relative to adjacent components is less than 2 millimeters. In an embodiment, wherein clearance above and below the rotating disc relative to adjacent components is less than 1 millimeter. In an embodiment, wherein clearance above and below the rotating disc relative to adjacent components is less than 0.5 millimeters.

In an embodiment, the rotor has a thickness of less than 5 millimeters. In an embodiment, the rotor has a thickness of less than 4 millimeters. In an embodiment, the rotor has a thickness of less than 3 millimeters. In an embodiment, the rotor has a thickness of less than 2 millimeters. In an embodiment, the thickness of the polymagnet are less than 2 millimeters. In an embodiment, the thickness of the polymagnet are less than 1 millimeter.

In an embodiment, the thickness of the polymagnet are arranged in a radially symmetric pattern.

In an embodiment, the polymagnet cover at least 50 percent of the surface area of the rotor. In an embodiment, the polymagnet cover at least 60 percent of the surface area of the rotor. In an embodiment, the polymagnet cover at least 70 percent of the surface area of the rotor. In an embodiment, the polymagnet cover at least 80 percent of the surface area of the rotor. In an embodiment, the polymagnet cover at least 90 percent of the surface area of the rotor. In an embodiment, the polymagnet cover at least 95 percent of the surface area of the rotor.

The present design allows for a reduction of mass by: (a) dramatic reduction of mass of the permanent rare earth; (b) reduction of size of windings, coils; and (c) the ability to make general reductions of all materials

The present design allows, in some embodiments, for a reduction in power consumption, allowing greater battery life and reduction in size of external power management systems

The present design allows, in some implementations, for greater proximities of coils to specific field locations on the magnet allows stronger pull forces which allow changes in geometries in the design, allowing more impeller features on the magnet

The present design allows, in some embodiments, for use of polymagnets that allow a magnet construction with additional features that will allow us to put the attract and repel forces in close proximity to the coils.

The present design allows, in some embodiments, for a reduction of blood sheer and resulting thrombus and ischemic events (primarily stroke).

The present design allows, in dome embodiments, for an impeller/magnet to become an assembly with greatly reduced part mass, allowing the assembly to be levitated in another axis such that the assembly is in levitation when the patient is in a horizontal (as when sleeping).

Now, in reference to the drawings:

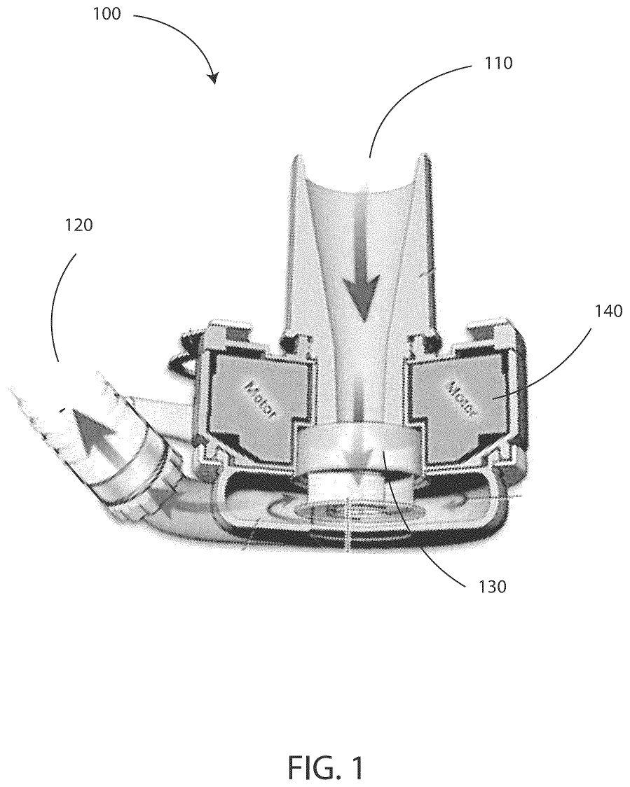

FIG. 1 is a simplified cross-sectional view of conventional left ventricle assist device 100, including an inlet 110 for receiving blood and outlet 120 for the blood. A motor assembly 140 is shown, along with a rotating disc and impeller assembly 130.

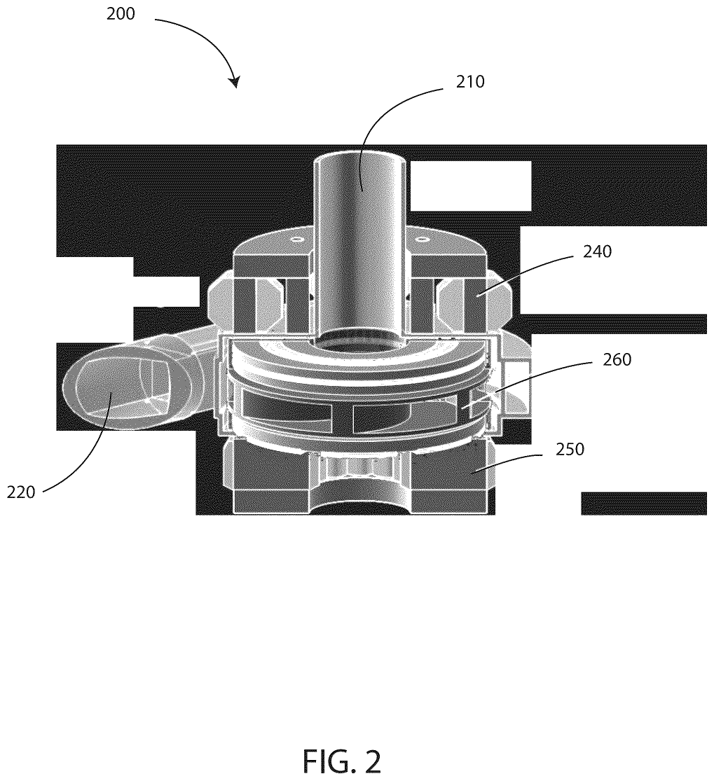

FIG. 2 is a simplified cross-sectional view of a conventional left ventricle assist device 200, including an inlet 110 for receiving blood and an outlet 220 for the blood. Also shown is a magnetic bearing stator 240, a magnetic stator 250, and a levitated impeller 260.

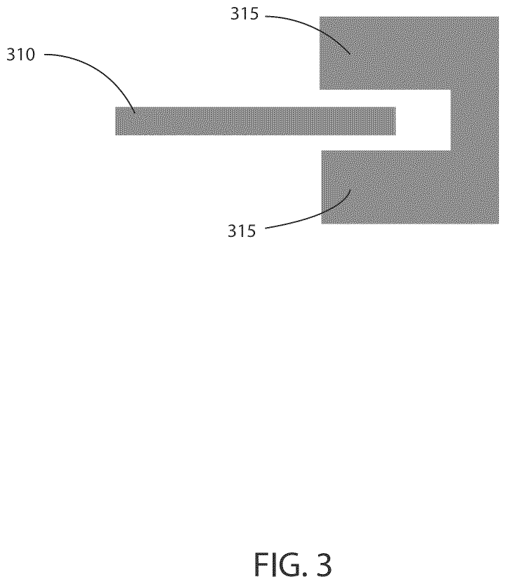

FIG. 3 is a cross-sectional schematic view of elements of left ventricle assist device in accordance with various embodiments herein, here showing levitation magnets 315 and rotating disc 310. In typical use there will be levitating magnets that perform a bearing function (allowing the disc to rotate with reduced friction), plus electric coils (not shown) that can engage alternating magnetic areas on the disc to cause it rotate (thereby providing the force to engage an impeller or other device to pressurize blood and pump it through the LVAD).

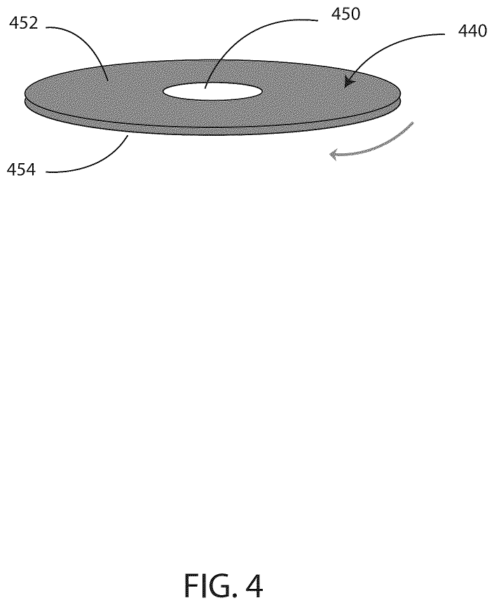

FIG. 4 is a perspective view of magnetic disc 440 in accordance with various embodiments herein, including an interior opening 450 (through which blood flows), and a top face 452 and bottom face 454. Top face 452 and bottom face 454 are magnetized using a polymagnets process to provide a custom, durable magnetic disc with positive/negative (north/south) regions for engaging coils to drive rotation of the disc and (optionally) to provide a levitating force.

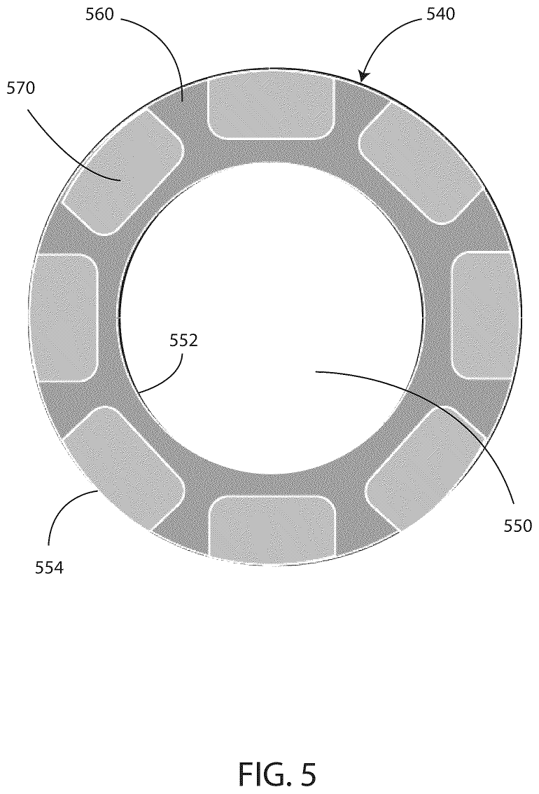

FIG. 5 is a top plan view of magnetic disc 540 in accordance with various embodiments herein, the magnetic disc 540 including an interior opening 550, the magnetic disc 540 interior opening 550 having an inner edge 552 and an outer edge 554. The magnetic disc 540 includes first area 560 having one magnetic polarity (such as “north” or “positive”) and second areas 570 having an opposite magnetic polarity (such as “south” or negative)”. In this embodiment the first area 560 extends around the entire 540, with eight distinct regions forming the second areas 570 having opposite magnetic polarity to the first area 560.

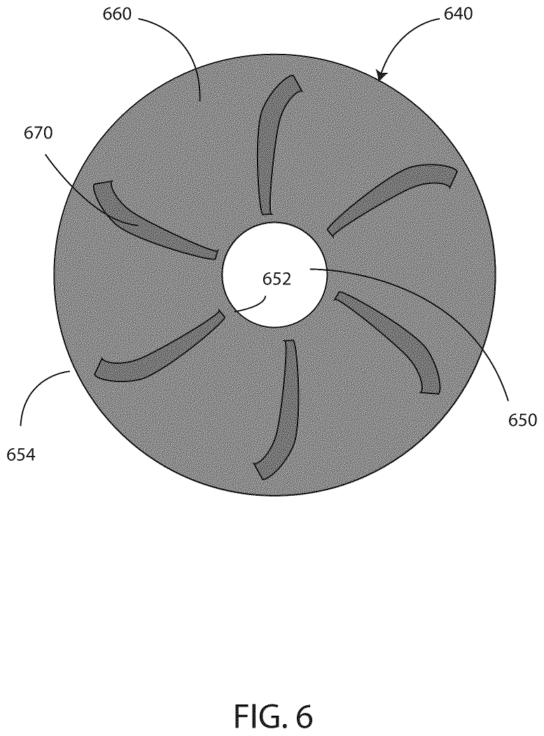

FIG. 6 is a top plan view of magnetic disc 640 in accordance with various embodiments herein, the magnetic disc 640 including an interior opening 650, the magnetic disc 640 interior opening 650 having an inner edge 652 and an outer edge 654. The magnetic disc 640 includes first area 660 having one magnetic polarity (such as “north” or “positive”) and second areas 670 having an opposite magnetic polarity (such as “south” or negative)”. In this embodiment the first area 660 extends around the entire 640, with six distinct regions forming the second areas 670 having opposite magnetic polarity to the first area 660. This embodiment also has a smaller interior opening 650 than the interior opening 550 of the magnetic disc 540.

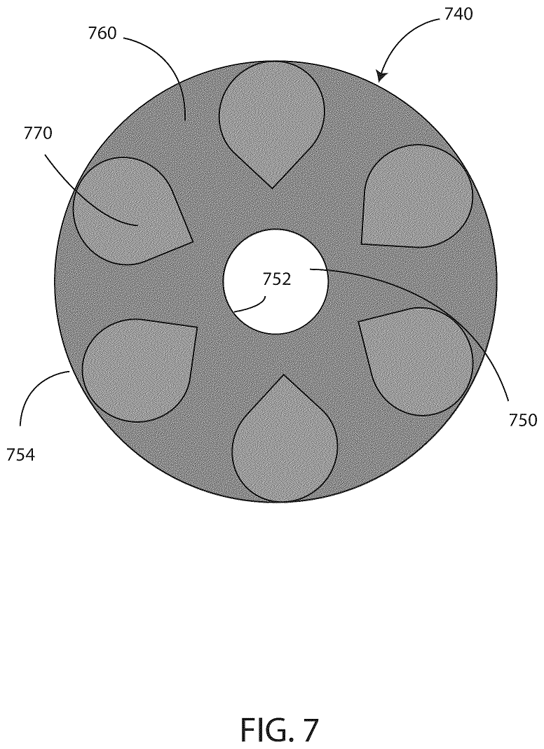

FIG. 7 is a top plan view of magnetic disc 740 in accordance with various embodiments herein, the magnetic disc 740 including an interior opening 750, the magnetic disc 740 interior opening 750 having an inner edge 752 and an outer edge 754. The magnetic disc 740 includes first area 760 having one magnetic polarity (such as “north” or “positive”) and second areas 770 having an opposite magnetic polarity (such as “south” or negative)”. In this embodiment the first area 760 extends around the entire 740, with six distinct “teardrop” regions forming the second areas 770 having opposite magnetic polarity to the first area 760.

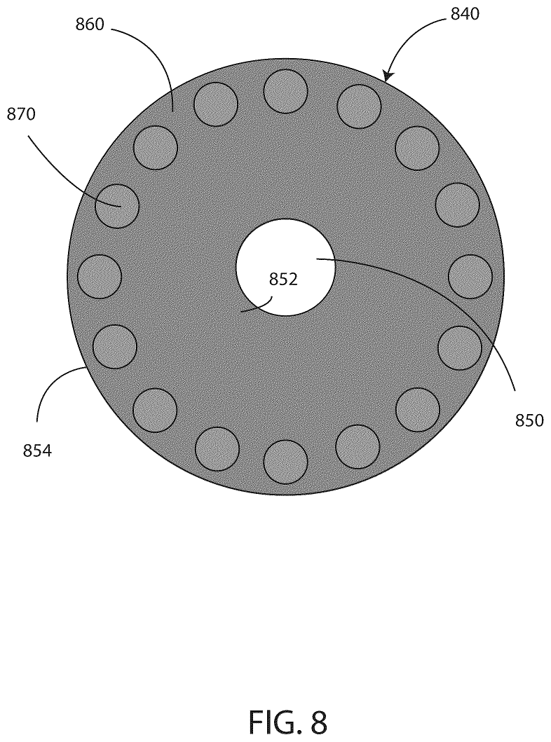

FIG. 8 is a top plan view of magnetic disc 840 in accordance with various embodiments herein, the magnetic disc 840 including an interior opening 850, the magnetic disc 840 interior opening 850 having an inner edge 852 and an outer edge 854. The magnetic disc 840 includes first area 860 having one magnetic polarity (such as “north” or “positive”) and second areas 870 having an opposite magnetic polarity (such as “south” or negative)”. In this embodiment the first area 860 extends around the entire 840, with sixteen distinct regions forming the second areas 870 having opposite magnetic polarity to the first area 860. The second areas 870 are positioned close to the outer edge 854 of the magnetic disc 840 so as to engage coils within an LVAD housing to impel rotation of the disc. The interior opening 850 is shown relatively small, but can be formed to be much larger (similar to FIG. 5).

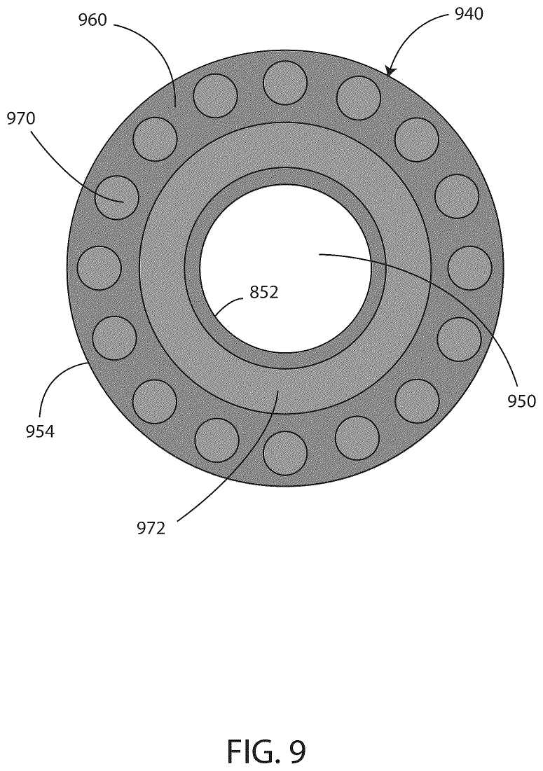

FIG. 9 is a top plan view of magnetic disc 940 in accordance with various embodiments herein, the magnetic disc 940 including an interior opening 950, the magnetic disc 940 interior opening 950 having an inner edge 952 and an outer edge 954. The magnetic disc 940 includes first area 960 having one magnetic polarity (such as “north” or “positive”) and second areas 970 having an opposite magnetic polarity (such as “south” or negative)”. In this embodiment the first area 960 extends around the entire 940, with sixteen distinct regions forming the second areas 970 having opposite magnetic polarity to the first area 960. In addition region 972 is magnetized w/the same polarity as second areas 970, and can be used

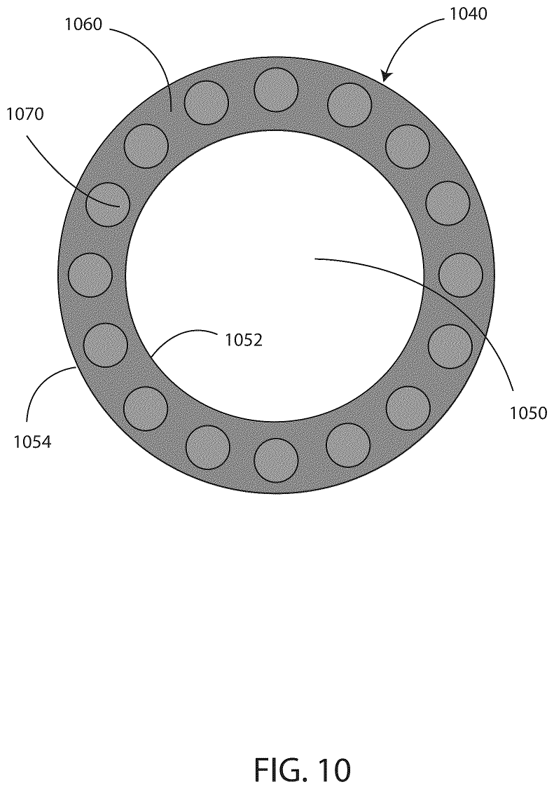

FIG. 10 is a top plan view of magnetic disc 1040 in accordance with various embodiments herein, the magnetic disc 1040 including an interior opening 1050, the magnetic disc 1040 interior opening 1050 having an inner edge 1052 and an outer edge 1054. The magnetic disc 1040 includes first area 1060 having one magnetic polarity (such as “north” or “positive”) and second areas 1070 having an opposite magnetic polarity (such as “south” or negative)”. In this embodiment the first area 1060 extends around the entire magnetic disc 1040, with sixteen distinct regions forming the second areas 1070 having opposite magnetic polarity to the first area 1060.

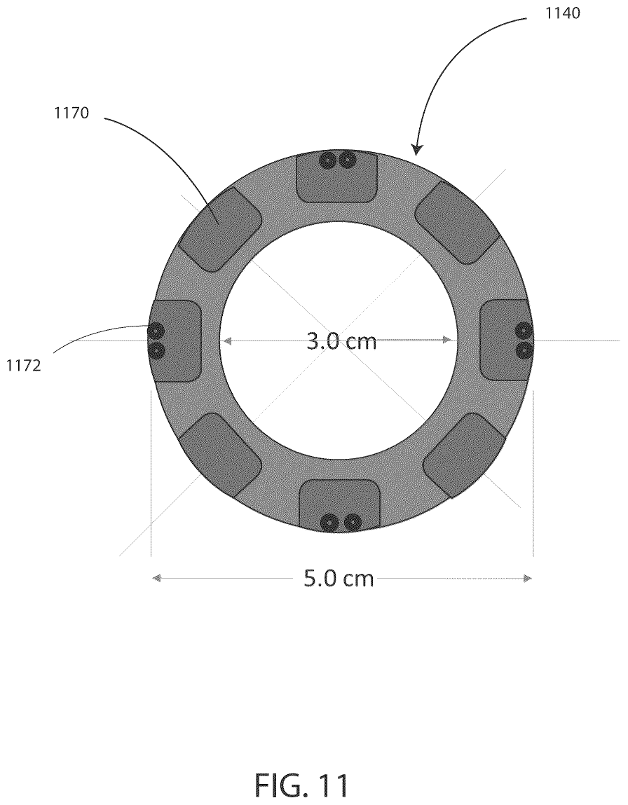

FIG. 11 is a top plan view of magnetic disc 1140 in accordance with various embodiments herein, showing area 1170 with a magnetic field, as well as sensors 1172.

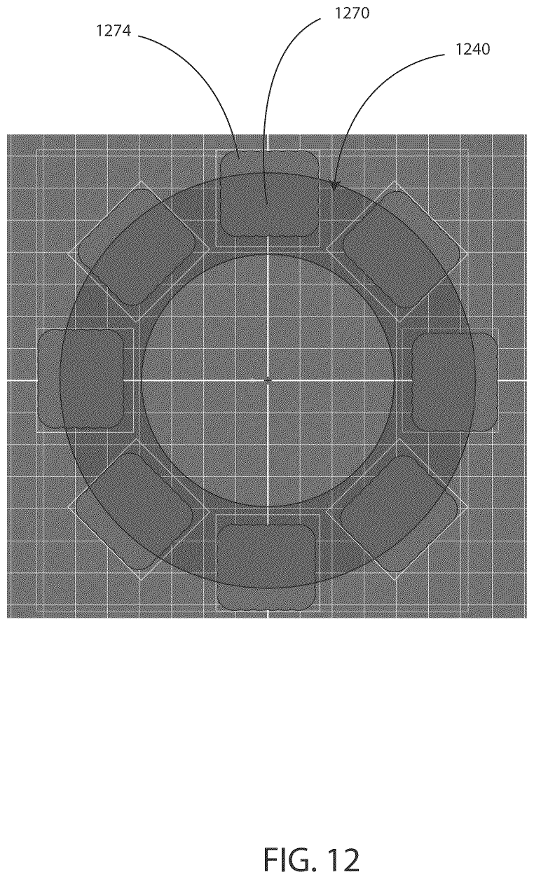

FIG. 12 is a top plan view of magnetic disc 1270 during manufacturing in accordance with various embodiments herein. FIG. 12 shows the disc 1270 with a magnetic area 1270 that will remain with the disc after manufacturing, along with an area 1274 that is removed during processing.

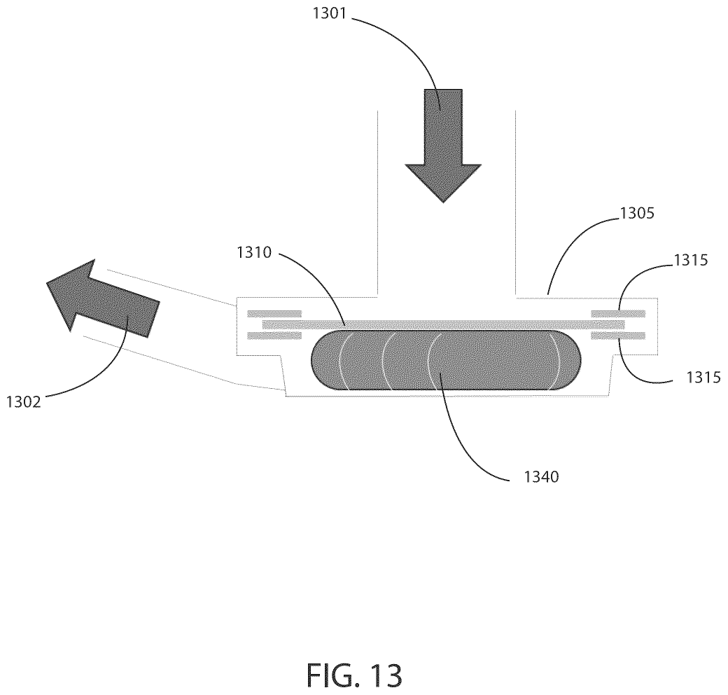

FIG. 13 is a cross-sectional schematic view of elements of left ventricle assist device in accordance with various embodiments herein, including an inlet 1301, an outlet 1302, a housing 1305, magnets 1315, disc 1310, and impeller assembly 1340.

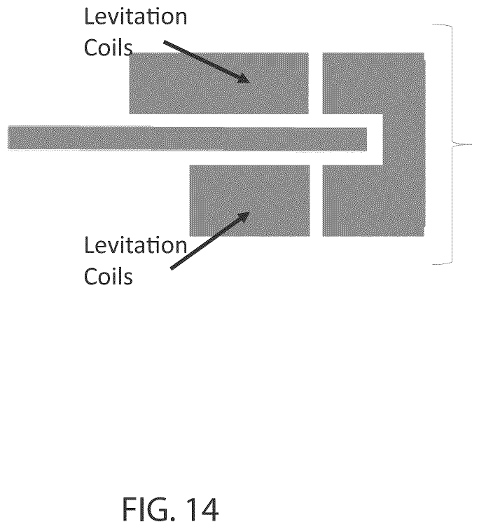

FIG. 14 cross-sectional schematic view of elements of left ventricle assist device in accordance with various embodiments herein, showing how levitation coils are positioned above and below a disc, and where drive coils are positioned on the outer circumference of the disc. Coils serve to both rotate and levitate the magnet/impeller assembly. They move the magnet in rotation by creating a temporary magnetic field of opposite polarities, above and below, to attract the fixed fields of the magnet. In addition, the strength of the field of a given coil is dynamically varied to continually position (“levitate”) the magnet/impeller assembly. This allows the magnet to levitate vertically, horizontally, and to be positioned dynamically in two ways: (a) centered in the device; (b) equidistant between the coils (“floating ”).

In an optimized version, the magnet is reduced in mass as much as possible by opening the center, since the primary function is at the outer area. In an optimized version, the magnet is affixed to the impeller and the impeller is the primary, mechanical structure.

Coils serve to both rotate and levitate the magnet/impeller assembly. They move the magnet in rotation by creating a temporary magnetic field of opposite polarities, above and below, to attract the fixed fields of the magnet.

In addition, the strength of the field of a given coil is dynamically varied to continually position (“levitate”) the magnet/impeller assembly. This allows the magnet to levitate vertically, horizontally, and to be positioned dynamically in two ways: (a) centered in the device; (b) equidistant between the coils (“floating”).

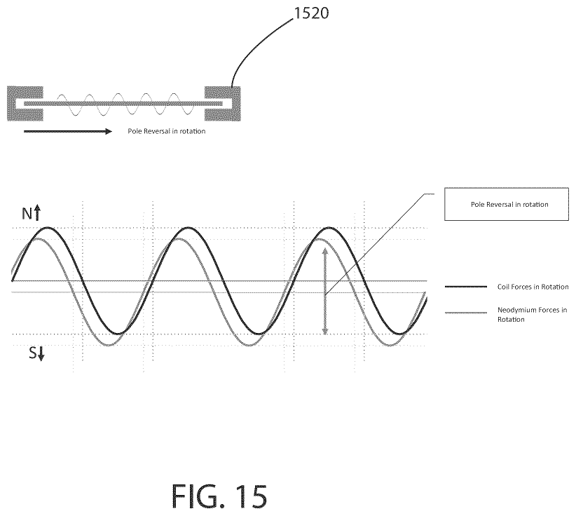

FIG. 15 are illustrations showing coil forces applied during operation of an LVAD in accordance with various embodiments herein, showing how the forces change, including pole reversal, to rotate the disc.

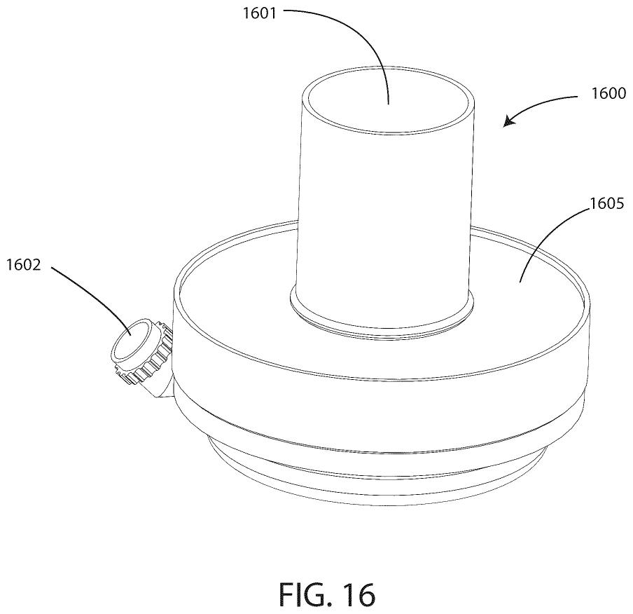

FIG. 16 is a perspective view of left ventricle assist device 1600 in accordance with various embodiments herein, showing an inlet 1601, outlet 1602 and body 1605.

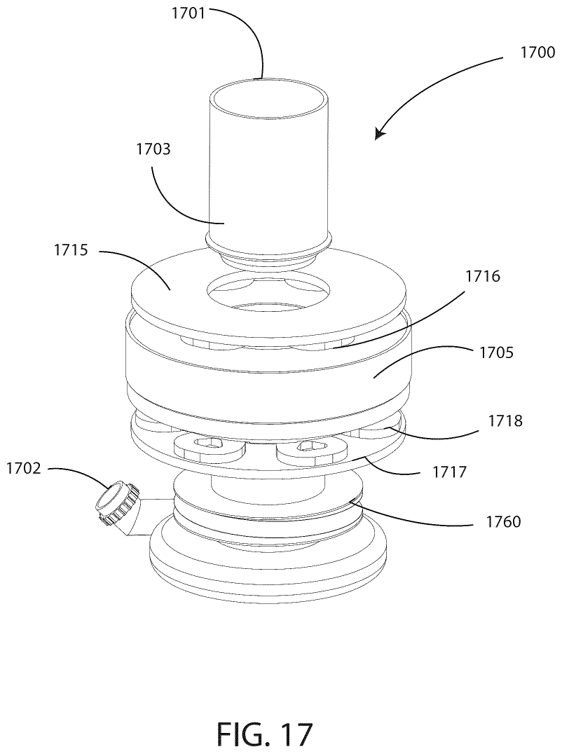

FIG. 17 is an exploded perspective view of left ventricle assist device 1700 in accordance with various embodiments herein, including an inlet 1701, outlet 1702, first disc 1715 and second disc 1717, a body housing 1705, coils 1716 and 1718, plus an impeller assembly 1760.

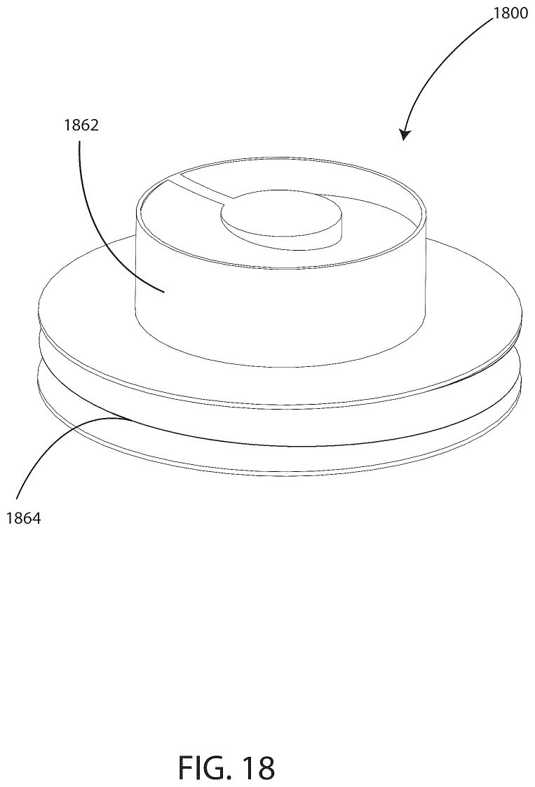

FIG. 18 is a perspective view of an impeller assembly 1800 for a left ventricle assist device in accordance with various embodiments herein, including a first portion 1862 and second portion 1864 with a greater diameter than the first portion. Generally it is desirable to reduce blood sheer by having a larger opening and larger impeller, with more impelling surface and reduce “edge length”.

The impeller can be made of UHMW-PE with coating of PTFE. An alternative is PEX. Two parts join together to capture the plastic parts to the magnet. UHMW has high molecular weight and can stand very high number of cycles. Alternatively titanium may be used.

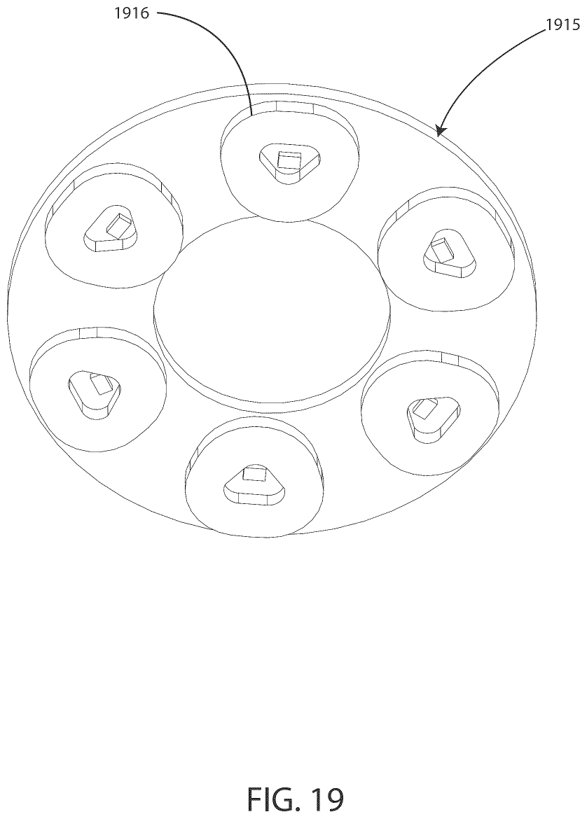

FIG. 19 is a perspective view of portions of a magnetic disc in accordance with various embodiments herein.

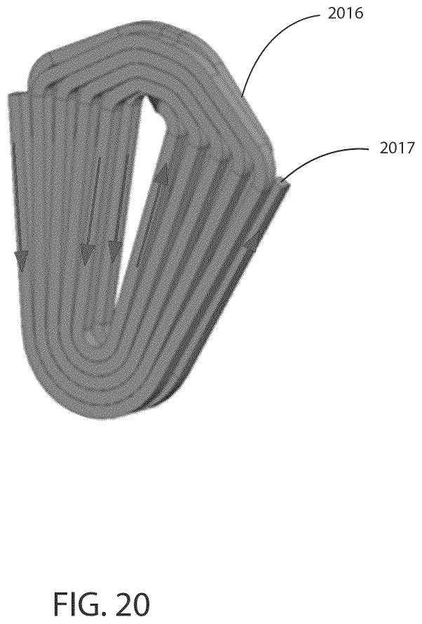

FIG. 20 is a perspective view of example coils from a magnetic disc in accordance with various embodiments therein.

The rotating discs described herein may be formed using the systems and processes termed “polymagnets” by Correlated Magnetics Research, LLC, including as described in U.S. Pat. Nos. 9,406,424; 8,570,129; 8,174,347; 9,082,539; 9,367,783; 8,844,412; 10,173,292; 8,872,608; 7,755,462; 9,105,384; 8,917,715; 8,937,521; and 10,194,246.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this application pertains. All publications and patent applications are herein incorporated by reference to the same extent as if each individual publication or patent application was specifically and individually indicated by reference.

The headings used herein are provided for consistency with suggestions under 37 CFR 1.77 or otherwise to provide organizational cues. These headings shall not be viewed to limit or characterize the application(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any application(s) in this disclosure. Neither is the “Summary” to be considered as a characterization of the application(s) set forth in issued claims.

The embodiments described herein are not intended to be exhaustive or to limit the application to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A left ventricle assist device (LVAD), the LVAD comprising:
an inflow port;
an outflow port;
an impeller configured to pump blood from the inflow port to the outflow port so as to assist the left ventricle of a heart in pumping blood; and
a motor driving the impeller, the motor comprising:
a stator element; and
a rotating disc element;
wherein the rotating disc element comprises a polymagnet.

2. The left ventricle (LVAD) assist device of claim 1, wherein the rotating disc element comprises a polymagnet configured for levitation.

3. The left ventricle (LVAD) assist device of claim 2, wherein the levitation magnets are positioned on one of two faces of the rotating disc.

4. The left ventricle (LVAD) assist device of claim 2, wherein the levitation magnets are positioned on two faces of the rotating disc.

5. The left ventricle (LVAD) assist device of claim 1, wherein the rotating disc element comprises a polymagnet configured for application of rotational force to the rotating disc.

6. The left ventricle (LVAD) assist device of claim 1, wherein the rotating disc comprises a polymagnet configured for application of rotational force and levitating force to the rotating disc.

7. The left ventricle (LVAD) assist device of claim 6, wherein the rotating disc comprises a polymagnet configured for application of rotational force and levitating force to the rotating disc;
wherein the rotating disc comprises a polymagnet for rotational force and levitating force on both sides of the rotating disc.

8. The left ventricle (LVAD) assist device of claim 7, wherein the rotating disc comprises a polymagnet configured for application of rotational force and levitating force to the rotating disc;
wherein the polymagnet for the rotational force is located radially distinct from the polymagnet for levitation relative to the center of the disc.

9. The left ventricle (LVAD) assist device of claim 7, wherein the rotating disc comprises a polymagnet configured for application of rotational force and levitating force to the rotating disc;
wherein the polymagnet for the rotational force is located concentrically outside relative to the polymagnet for levitation.

10. The left ventricle (LVAD) assist device of claim 1, wherein the rotating disc comprises a polymagnet configured for application of rotational force, wherein the polymagnet comprise alternating positive and negative magnetic regions.

11. The left ventricle (LVAD) assist device of claim 1, wherein the rotating disc comprises a polymagnet configured for application of levitational force, wherein the polymagnet comprise continuous concentric regions of positive or negative magnetic polarity.

12. The left ventricle (LVAD) assist device of claim 1, wherein the polymagnet comprises sintered NiCuNi material.

13. The left ventricle (LVAD) assist device of claim 1, wherein the polymagnet comprises NdFeB material.

14. The left ventricle (LVAD) assist device of claim 1, wherein the polymagnet comprises sintered SmCo material.

15. The left ventricle (LVAD) assist device of claim 1, wherein the polymagnet comprise a PTFE material.

16. The left ventricle (LVAD) assist device of claim 1, wherein the rotating disc comprises an open interior for passage of blood, the open interior having an area at least 10 percent of the area within the circumference of the disc.

17. The left ventricle (LVAD) assist device of claim 1, wherein the rotating disc comprises an open interior for passage of blood, the open interior having an area at least 15 percent of the area within the circumference of the disc.

18. The left ventricle (LVAD) assist device of claim 1, wherein the rotating disc comprises an open interior for passage of blood, the open interior having an area at least 20 percent of the area within the circumference of the disc.

19. The left ventricle (LVAD) assist device of claim 1, wherein the rotating disc comprises an open interior for passage of blood, the open interior having an area at least 25 percent of the area within the circumference of the disc.

20. The left ventricle (LVAD) assist device of claim 1, wherein the rotating disc comprises an open interior for passage of blood, the open interior having an area at least 30 percent of the area within the circumference of the disc.

21.-43. (canceled)
